(19) United States
(12) Patent Application Publication
(10) Pub. No.: US 2025/0018334 A1
(43) Pub. Date: Jan. 16, 2025

(54) FLUE GAS STACK SUCTION CONTROL

(71) Applicant: AKER CARBON CAPTURE NORWAY AS, LYSAKER (NO)

(72) Inventor: PETTER VANGSNES, SNAROEYA (NO)

(73) Assignee: AKER CARBON CAPTURE NORWAY AS, LYSAKER (NO)

(21) Appl. No.: 18/712,722

(22) PCT Filed: Nov. 23, 2022
(86) PCT No.: PCT/NO2022/050270

§ 371 Date: May 23, 2024

(30) Foreign Application Priority Data
Nov. 23, 2021 (NO) 20211394

(51) Int. Cl. B01D 53/34; F23J 15/02

(52) U.S. Cl. CPC B01D 53/346; F23J 15/02; B01D 2258/0283

(57) ABSTRACT

An industrial plant includes a flue gas processing system, a stack which receives a flue gas from an industrial process, a flue gas processing unit, a suction line connected between the stack and the flue gas processing unit, a fan arranged in the suction line, a sensor which provides a signal representative of a gas flow rate or a change thereof through a discharge outlet of the stack, a controller which regulates a flow through the suction line based on the signal, and a flow restriction arranged in the suction line upstream of the fan. The discharge outlet is open to the atmosphere. The fan draws the flue gas from the stack to the flue gas processing unit and has a control arrangement which controls a pressure in the suction line upstream of the fan and downstream of the flow restriction or a pressure differential across the flow restriction.

This application is a U.S. National Phase application under 35 U.S.C. § 371 of International Application No. PCT/NO2022/050270, filed on Nov. 23, 2022 and which claims benefit to Norwegian patent application No. 20211394, filed on Nov. 23, 2021. The International Application was published in English on Jun. 1, 2023 as WO 2023/096497 A1 under PCT Article 21(2).

### FIELD

The present invention relates to a system and to a method for control of flue gas handling from an industrial plant into a flue gas treatment plant.

### BACKGROUND

In industrial plants, such as power plants or other types of plants producing flue gases, there is often a need to process the flue gases before discharge to the environment. This may, for example, be the case if it is required or desirable to clean or remove parts from the flue gas stream. One relevant example in this respect is CO₂ capture from flue gases in order to reduce CO₂ emissions.

### SUMMARY

In an embodiment, the present invention provides an industrial plant which includes a flue gas processing system. The industrial plant comprises a flue gas stack which is configured to receive a flue gas from at least one industrial process, a flue gas processing unit, a suction line which is connected between the flue gas stack and the flue gas processing unit, a fan which is operatively arranged in the suction line, a sensor which is configured to provide a signal which is representative of a gas flow rate or a change in the gas flow rate through a discharge outlet of the flue gas stack, a controller which is arranged to regulate a flow through the suction line based on the signal from the sensor, and a flow restriction arranged in the suction line upstream of the fan. The discharge outlet of the flue gas stack is open to the atmosphere. The fan is configured to draw the flue gas from the flue gas stack to the flue gas processing unit. The signal is a measured signal or a calculated signal. The fan comprises a control arrangement which is configured to control a pressure in the suction line upstream of the fan and downstream of the flow restriction or to control a pressure differential across the flow restriction.

### BRIEF DESCRIPTIONS OF THE DRAWINGS

The present invention is described in greater detail below on the basis of embodiments and of the drawings in which:

FIG. 1 shows a schematic illustration of an industrial plant having a flue gas processing system;

FIG. 2 shows a schematic illustration of an industrial plant having a flue gas processing system;

FIG. 3 shows a schematic illustration of an industrial plant having a flue gas processing system; and

FIG. 4 shows a schematic illustration of an industrial plant having a flue gas processing system.

### DETAILED DESCRIPTION

The present invention provides an industrial plant having a flue gas processing system. The industrial plant comprises a flue gas stack arranged to receive flue gases from an industrial process, the stack having a discharge outlet open to the atmosphere, a suction line connected to the flue gas stack and to a flue gas processing unit, a fan operatively arranged in the suction line, a sensor, and a controller arranged to regulate the flow through the suction line based on a measured signal from the sensor.

The present invention also provides a method of controlling the operation of an industrial plant having a flue gas processing system. The method comprises the steps of operating an industrial process to produce a flue gas, flowing the flue gas to a flue gas processing unit via a suction line which is fluidly connected to a flue gas stack having a discharge outlet open to the atmosphere, and operating a controller to control the flow rate of flue gas through the suction line in response to a measured signal.

The above, as well as additional features and advantages of the present invention, will be more fully appreciated by reference to the following illustrative and non-limiting detailed description of example embodiments of the present disclosure, when taken in conjunction with the accompanying drawings.

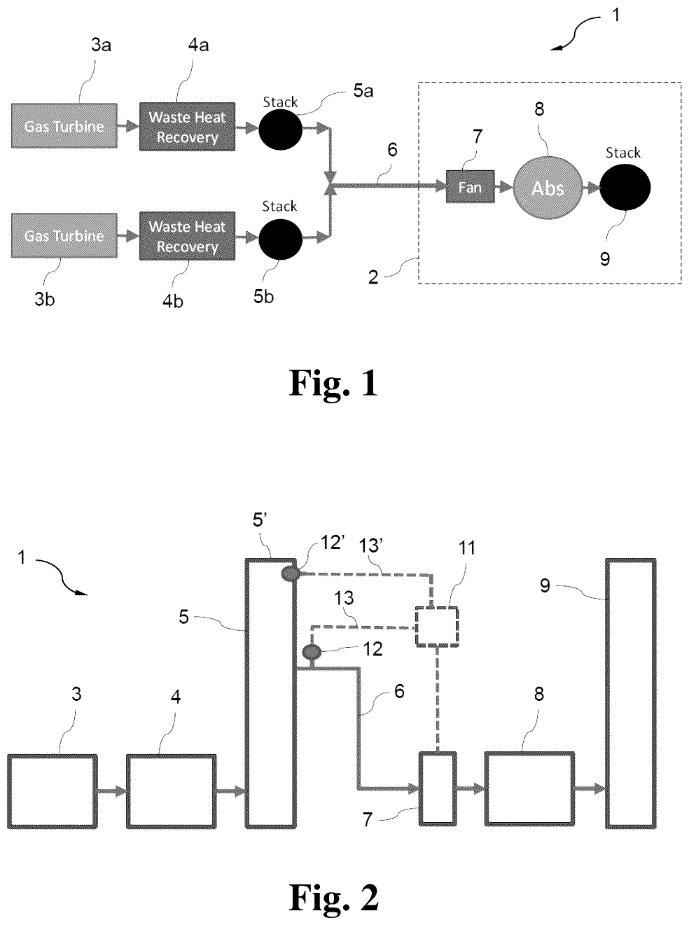

FIGS. 1 and 2 illustrate, in a schematic manner, an industrial plant 1 having a flue gas processing system 2. The flue gas processing system 2 is configured to receive flue gases from an industrial process, here illustrated as a gas turbine 3, 3a-b. The gas turbine 3, 3a-b produces flue gases (exhaust) which are optionally led through a waste heat recovery heat exchanger 4, 4a-b and subsequently to a flue gas stack 5, 5a-b. As can be seen from FIGS. 1 and 2, in FIG. 1, two industrial processes are illustrated in the form of two gas turbines 3a-b, while in FIG. 2, only one gas turbine 3 is illustrated. The industrial plant 1 may alternatively have more than two industrial processes, or may have two or more industrial processes of different types, such as different types of power plants producing a flue gas.

In FIG. 1, the two gas turbines 3a, 3b each have a dedicated flue gas stack 5a, 5b, however, two or more industrial processes, such as gas turbines 3a, 3b, may alternatively be connected to a common flue gas stack 5, 5a-b.

The flue gas stack 5, 5a-b is arranged to receive flue gases from the industrial process(es), in this case, from the gas turbine(s) 3, 3a-b via the waste heat recovery heat exchanger 4, 4a-b, and the flue gas stack 5, 5a-b which has a discharge outlet 5′ (see FIG. 2) open to the atmosphere.

A suction line 6 is connected to the flue gas stack 5, 5a-b and to a flue gas processing unit 8 so that the suction line 6 can transfer flue gases from the flue gas stack 5, 5a-b to the flue gas processing unit 8. The suction line 6 may be in the form of a duct, however, the suction line 6 may alternatively be formed by any suitable flow channel or pipe. A fan 7 is operatively arranged in the suction line 6 for generating a flow of flue gas to the flue gas processing unit 8. The fan 7 is controllable via a controller 11 so as to control the flow rate through the suction line 6 and/or the pressure(s) in the suction line 6. In this manner, a flue gas flow rate out of the flue gas stack 5, 5a-b through the suction line 6 can be generated and/or controlled. It may, for example, often be desirable to control this flow rate so that substantially all the flue gas from the industrial process is led to the flue gas processing unit 8 for treatment. This means that the flow at the discharge outlet 5′ is substantially zero and that the stack is being “balanced”. (This is hereafter referred to as “stack balancing control”.) It may alternatively be an objective to maintain a supply to the flue gas processing unit 8 which matches its treatment capacity in order to maximize utilization and/or to provide stable operating conditions for the flue gas processing unit 8.

The flue gas processing unit 8 may, for example, be a CO₂ removal plant.

In some applications, there may be a need for the flue gas stack 5, 5a-b to remain permanently open to atmosphere in order to prevent pressure build-up and disturbance of the industrial process. As the flue gas stack 5, 5a-b, and thus also the suction line 6, must then have an open connection to atmosphere via the flue gas stack 5, 5a-b (specifically, via the discharge outlet 5′), the pressure levels in the stack 5, 5a-b and the suction line 6 will be close to the atmospheric pressure, generally a few mbar below the atmospheric pressure to allow flow into and through the suction line 6. (1 atm=approximately 1.01325 bara).

As the pressure at the discharge outlet 5′ may vary with rapid variations in wind velocities around the flue gas stack 5, 5a-b and the industrial plant 1, the pressure in the flue gas stack 5, 5a-b may, however, in certain applications experience dynamic variations. This can be particularly prevalent in offshore plants where wind velocities can be higher, and/or in areas which experience more gusts and variations in wind conditions around the industrial plant 1. If using pressure levels in the flue gas stack 5, 5a-b and/or suction line 6 as a control parameter in the system, for example, to control the pressure levels or flow rates, poorer control performance may be experienced due to such disturbances.

Referring to the embodiment illustrated in FIG. 2, a controller 11 is arranged to regulate the flow through the suction line 6 based on a signal 13 from a temperature sensor 12 and indicative of a temperature or temperature change in the suction line 6. In FIG. 2, the controller 11 is arranged to regulate an operating speed of the fan 7 based on the signal 13 indicative of a temperature or temperature change in the suction line 6. In this manner, a cooling effect and/or sudden temperature change which may follow if the flue gas stack 5,5a-b starts sucking in colder air with ambient temperature via the discharge outlet 5′ can be rapidly identified, and appropriate control action can be taken. The flue gas will normally be more than 200° C., only a small amount of atmospheric air entering the flue gas flowing into the suction line 6 will therefore create significant temperature drops which are detectable via the temperature sensor 12. The suction rate through the suction line 6 can be adjusted in this manner so that no ambient air is drawn into the flue gas processing unit 8 via the discharge outlet 5′. This control setup therefore allows the flow rate of flue gas provided to the flue gas processing unit 8 to be adjusted according to variations in the supply rate of flue gas coming from the industrial process (here, the gas turbine 3), and thereby reduce the risk that the flue gas processing unit 8 is unintentionally supplied with flue gas which is diluted with ambient air. When such a change in temperature is detected, the flowrate from the flue gas stack 5, 5a-b into the suction line 6 and further to the flue gas processing unit 8 can thus be reduced until the temperature is re-normalized by not sucking in air via the discharge outlet 5′.

The temperature sensor 12 is illustrated in FIG. 2 as being placed near an inlet part of the suction line 6. The temperature sensor 12 may alternatively be placed in the flue gas stack 5, 5a-b, for example, in a position in the flue gas stack 5, 5a-b which is close to the suction line 6 connection, or at another position in the flue gas stack 5, 5a-b which would experience a temperature change if ambient air is drawn into the flue gas stack 5, 5a-b.

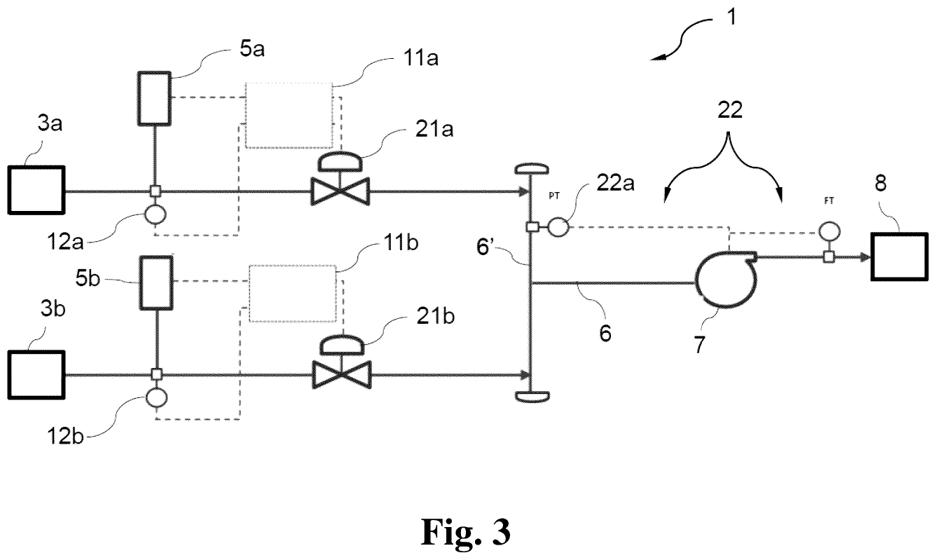

FIG. 3 illustrates another embodiment having several components which are equivalent to those illustrated in FIGS. 1 and 2, which are given the same reference numerals. In FIG. 3, the controllers 11a-b are operatively connected to variable flow restrictions 21a-b in the suction line 6. The flow restrictions 21a-b may, for example, be control dampers arranged in the suction line 6 which are operable to vary the flow resistance and/or effective cross-sectional flow area in the suction line 6. Butterfly dampers are one type of common flow restrictions which may be suitable for this purpose.

In response to a reduction in flue gas temperature measured by temperature sensor 12a, the controller 11a can then be configured to reduce the flow rate by adjusting the flow restriction 21a. Controller 11b can similarly be configured to regulate the flue gas flow rate via flow restriction 21b.

A suitable pressure is maintained in the suction line 6 by the fan 7 so as to allow a flow of flue gases to the flue gas processing unit(s) 8. This requires there to be a negative pressure differential across the flow restriction 21a/b so that the fluid pressure downstream of the flow restriction 21a/b is lower than the pressure upstream of the flow restriction 21a/b.

This could be achieved by setting the fan 7 to operate at a rate so that it will always create such a pressure differential when the gas turbines 3a-3b are in operation, whatever the extent to which the flow restrictions 21a/21b must be operated to restrict flow along the suction line 6. This is likely, however, to result in the fan 7 operating at much higher speeds than is actually needed to generate the required negative pressure differential for much of the time, and thus the energy being consumed by the process being much higher than it needs to be.

As such, in this embodiment, the fan 7 has its own control arrangement for this purpose, as indicated at 22, in order to maintain a desirable pressure in the suction line 6. The control arrangement 22 may, for example, be a standard feedback control loop regulating the speed of the fan 7 based on a reading from a pressure sensor 22a. The control arrangement 22 is configured to maintain the pressure at the pressure sensor 22a at a set level which is pre-determined so that the pressure downstream of the flow restrictions 21a/21b is always lower than the pressure in the flue gas stacks 3a, 3b based on an assessment of the likely range of the pressure in the flue gas stacks 3a, 3b when the gas turbines 3a, 3b are operational. By controlling the speed of the fan 7 based on the downstream pressure measurement, the speed of the fan 7 will be reduced when the flow restrictions 21a/21b are significantly restricting the flow of gas to the suction line 6, and increased when the degree of flow restriction is decreased.

The pressure sensor 22a providing the control signal for the control arrangement 22 is advantageously arranged in a manifold 6′ which is part of the suction line 6. The flue gas flow control is then effectuated by adjusting the flow restrictions 21a-b, while the fan 7 is controlled to maintain the desired pressure in the suction line 6.

This could again result in the fan 7 operating at higher speeds than is actually required to generate the required negative pressure differential for much of the time, in particular when the pressure in the flue gas stacks 3a, 3b is at the higher end of the expected pressure range, and thus the energy being consumed by the process being much higher than it needs to be. When the pressure in the flue gas stacks 5a, 5b is at the higher end of the expected range, the required pressure differential across the flow strictions 21a, 21b can be generated with a higher suction line pressure than when the pressure in the flue gas stack 5a, 5b is at the lower end of the expected range. To address this, additional pressure sensors may be provided to measure the pressure upstream of each of the flow restrictions 21a, 12b. These additional pressure sensors could be provided in the flue gas stacks 5a, 5b, at the inlet part of the suction 6 with the temperature sensors 12a, 12b, or directly adjacent the flow restrictions 21a, 21b. The control signals for the control arrangement 22 are in this case provided by the pressure sensor 22a and the additional upstream pressure sensors, and the control arrangement 22 is configured to regulate the speed of the fan 7 based on the pressure differential across the flow restrictions 21a, 21b as determined from the readings from these pressure sensors, and to maintain the pressure differential at a predetermined set level.

It will be appreciated that because the suction line 6 draws flue gas from two different flue gas stacks 5a, 5b in this embodiment, that the pressure differential across the two flow restrictions 21a, 21b may be different depending on the pressure in each of the flue gas stacks 5a, 5b. The control arrangement 22 will therefore be configured to control the speed of the fan 7 so that there is a negative pressure differential across both flow restrictions 21a, 21b. For example, if both flow restrictions 21a, 21b are restricting the flow along the suction line 6 to a similar extent, this will mean that the fan 7 speed is set based on the pressure differential across whichever of the flow restrictions 21a, 21b is connected to the flue gas stack 5a, 5b at the lowest pressure.

FIG. 3 illustrates two flue gas sources (here, gas turbines 3a-b), however, the industrial plant 1 may comprise a single industrial process making up a flue gas source, or more than two industrial processes making up flue gas sources.

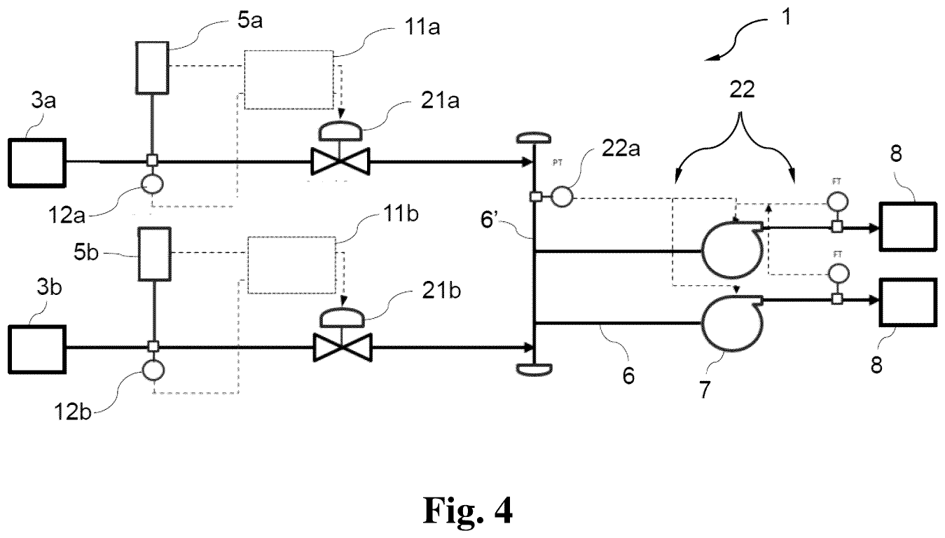

FIG. 4 illustrates a similar industrial plant 1 as shown in FIG. 3, but with two flue gas processing units 8. Each flue gas processing unit 8 may have its own fan 7. The embodiment in FIG. 4 makes up an alternative implementation to that shown in FIG. 3 and otherwise functions in the same way as described above. Yet further flue gas processing units 8 may optionally be used. Advantageously, when having more than one industrial process and/or more than one flue gas processing unit 8, each industrial process and each flue gas processing unit 8 can be fluidly connected to the manifold 6′ so that the manifold 6′ receives and distributes all flue gas in the system.

In alternative embodiments, the temperature sensor(s) 12, 12a-b described above may be a different type of sensor. In any of the embodiments described herein, the sensor(s) 12, 12a-b may in particular be (an) oxygen sensor(s) operable to measure an oxygen content of the gas stream passing by the sensor(s) 12, 12a-b. A measured increase in oxygen content can be taken as an indication of influx of atmospheric air through the discharge outlet 5′. Control actions similar to those described above may be initiated based on such a sensor signal. Alternatively, in any of the embodiments described herein, the sensor(s) 12, 12a-b may be combined temperature and oxygen sensor(s), whereby control action is taken by the controller 11 on the basis of both a temperature signal and an oxygen level signal. This can increase accuracy or otherwise improve control performance in some embodiments.

Alternatively or additionally, the industrial plant 1 may comprise a flow sensor 12′ such as one or more flowmeter(s)) arranged in the flue gas stack 5. The flow sensor 12′ (see FIG. 2) produces a signal 13′ indicative of a gas flow rate through (i.e., into and/or out of) the discharge outlet 5′. The signal 13′ can be provided to the controller 11 and used to generate control actions similar to those described above. In any of the embodiments described herein, the industrial plant 1 can use a flow sensor 12′ and a signal 13′ thereof in conjunction with a signal 13, 13′ from the sensor(s) 12, 12a-b or independently, i.e., without the sensor(s) 12, 12a-b.

In any of the embodiments described herein, the sensor may be arranged to provide a measured signal 13, 13′ or a calculated signal which is representative of a gas flow rate or change in gas flow rate through the discharge outlet 5′. The signal may be a direct measurement signal, a combination of measurement signals (e.g., for different operational parameters), or a calculated signal, for example, calculated based on a model or look-up tables.

In any of the embodiments described herein, the controller 11 can be configured to regulate the flow through the suction line 6 so that the flow rate through the discharge outlet 5′ remains at zero, close to zero, or with a positive outflow of gas from the flue gas stack 5 through the discharge outlet 5′. An inflow of atmospheric air through the discharge outlet 5′ and into the flue gas processing unit 8 can thereby be avoided, or at least the risk thereof can thereby be reduced. Alternatively, in some cases, it may be acceptable that some atmospheric air reaches the flue gas processing unit 8 but it is more important to avoid untreated flue gas to exit to the atmosphere. The controller 11 can in such a case be configured to regulate the flow through the suction line 6 so that the flow rate through the discharge outlet 5′ remains at or close to zero, or with some inflow of gas from the flue gas stack 5 through the discharge outlet 5′.

The sensor(s) used in the industrial plant 1 may be of any suitable type for measuring temperature, oxygen and/or flow. In one embodiment, the temperature sensor may be an IR-camera operable to generate an image or heat map measuring the temperature distribution of gas at a location in the flue gas stack 5 or the suction line 6. The image or heat map may, for example, be generated on an outside surface of the stack, and thereby reflect a change in gas temperature on the inside of the stack. A control signal 13, 13′ can then be calculated based on the image, for example, by generating a parameter, for example, based on the lowest temperature visible in the image or map, or based on a calculated average temperature across defined point in (or the entire) image or map.

According to embodiments described herein, an open flue gas suction system is provided with improved handling of incoming flue gas from a stack, and with a lower risk of pulling in ambient air, so as to provide a robust control that can handle wind gust and high wind velocities around the stack creating variation in the outside air pressure at the stack outlet.

The present invention is not limited to embodiments described herein; reference should be had to the appended claims.

### LIST OF REFERENCE NUMERALS

1 Industrial plant
2 Flue gas processing system
3 Gas turbine
3a-b Gas turbine
4 Waste heat recovery heat exchanger
4a-b Waste heat recovery heat exchanger
5 Flue gas stack
5a-b Flue gas stack
5′ Discharge outlet
6 Suction line
6′ Manifold
7 Fan
8 Flue gas processing unit
9 Stack
11 Controller
11a-b Controllers
12 Sensor/Temperature sensor
12a-b Sensor/Temperature sensor
12′ Flow sensor
13, 13′ Signal/Control signal/Measured signal
21a-b Flow restriction
22 Control arrangement
22a Pressure sensor

What is claimed is:

1-19. (canceled)

20. An industrial plant comprising a flue gas processing system, the industrial plant comprising:
a flue gas stack which is configured to receive a flue gas from at least one industrial process, the flue gas stack comprising a discharge outlet which is open to the atmosphere;
a flue gas processing unit;
a suction line which is connected between the flue gas stack and the flue gas processing unit;
a fan which is operatively arranged in the suction line, the fan being configured to draw the flue gas from the flue gas stack to the flue gas processing unit;
a sensor which is configured to provide a signal which is representative of a gas flow rate or a change in the gas flow rate through the discharge outlet of the flue gas stack, the signal being a measured signal or a calculated signal;
a controller which is arranged to regulate a flow through the suction line based on the signal from the sensor; and
a flow restriction arranged in the suction line upstream of the fan,
wherein,
the fan comprises a control arrangement which is configured to control a pressure in the suction line upstream of the fan and downstream of the flow restriction or to control a pressure differential across the flow restriction.

21. The industrial plant as recited in claim 20, wherein the measured signal from the sensor is indicative of at least one of,
a gas temperature or a change in the gas temperature in the flue gas stack or in the suction line,
an oxygen content or a change in the oxygen content in the flue gas stack or in the suction line, and
a gas flow rate or a change in the gas flow rate through the discharge outlet.

22. The industrial plant as recited in claim 20, wherein the control arrangement is further configured to maintain the pressure in the suction line upstream of the fan and downstream of the flow restriction at a substantially constant level.

23. The industrial plant as recited in claim 20, wherein the control arrangement is further configured to maintain the pressure differential across the flow restriction at a substantially constant level.

24. The industrial plant as recited in claim 20, further comprising:
a plurality of the at least one industrial process,
wherein,
the flow restriction arranged in the suction line is associated for each industrial process, and
the flow restriction is further configured to regulate the flue gas stream from a respective industrial process independently of any other industrial process in the industrial plant.

25. The industrial plant as recited in claim 24, further comprising:
a manifold which is configured to receive and to distribute all flue gas in the flue gas processing system,
wherein,
each industrial process and the flue gas processing unit are connected to the manifold.

26. The industrial plant as recited in claim 25, wherein,
the plurality of the at least one industrial process comprises a first industrial process and a second industrial process,
the flow restriction arranged in the suction line comprises a first flow restriction and a second flow restriction,
the first flow restriction is associated with the first industrial process, is arranged in the suction line upstream of the manifold, and is configured to direct the flue gas from the first industrial process to the manifold,
the second flow restriction is associated with the second industrial process, is arranged in the suction line upstream of the manifold, and is configured to direct the flue gas from the second industrial process to the manifold, and
the fan which is operatively arranged in the suction line is arranged downstream of the manifold, and is configured to draw the flue gas from both the first industrial process and from the second industrial process to the flue gas processing unit.

27. The industrial plant as recited in claim 20, wherein the controller is further configured to maintain a zero gas flow rate through the discharge outlet or to maintain a gas flow rate out of the flue gas stack through the discharge outlet.

28. The industrial plant as recited in claim 20, wherein the flue gas processing unit is a CO₂ removal plant.

29. A method of controlling an operation of an industrial plant which comprises a flue gas processing system, the method comprising:
operating an industrial process to produce a flue gas;
flowing the flue gas from a flue gas stack to a flue gas processing unit via a suction line, the flue gas stack comprising a discharge outlet which is open to the atmosphere;
operating a controller to control a flow rate of the flue gas through the suction line by regulating a flow restriction in the suction line in response to a signal from a sensor, the signal being a measured signal or a calculated signal which is representative of a gas flow rate or a change in the gas flow rate through the discharge outlet of the flue gas stack; and
operating a control arrangement of a fan, the fan being arranged in the suction line downstream of the flow restriction and being configured to control a pressure in the suction line downstream of the flow restriction and upstream of the fan or to control a pressure differential across the flow restriction.

30. The method as recited in claim 29, wherein the measured signal from the sensor is indicative of at least one of,
a gas temperature or a change in the gas temperature in the flue gas stack or in the suction line,
an oxygen content or a change in the oxygen content in the flue gas stack or in the suction line, and
a gas flow rate through the discharge outlet.

31. The method as recited in claim 29, wherein the method further comprises:
operating the controller to maintain a zero gas flow rate through the discharge outlet or to maintain a gas flow rate out of the flue gas stack through the discharge outlet.
